SEQUENCE LISTING (3 sequences)

SEQ ID NO: 1 - DNA, 48 bp, Escherichia coli
ctgcttctcatctgttgtcaacgccttgacgaaggtgtgagtcgtggt

SEQ ID NO: 2 - DNA, 24 bp, Escherichia coli
  primer_bind (1)..(24)
cgactcctggagcccgtcagtatc

SEQ ID NO: 3 - DNA, 24 bp, Escherichia coli
  primer_bind (1)..(24)
gaccactgatatcctgtctttaac

(19) United States
(12) Patent Application Publication
(10) Pub. No.: US 2004/0018619 A1
(43) Pub. Date: Jan. 29, 2004

(54) Immortalized lines of endothelial brain cells and therapeutic applications thereof

(76) Inventors: Nathalie Chaverot, Paris (FR); Pierre-Olivier Couraud, Auffargis (FR); John J. Laterra, Baltimore, MD (US); Jerome Quinonero, Noisiel (FR); Francoise Roux, Paris (FR); Arthur Donny Strosberg, Paris (FR); Jean-Leon Tchelingerian, Gif-Sur-Yvette (FR); Lionel Vignais, Paris (FR)

Correspondence Address: MINTZ LEVIN COHN FERRIS GLOVSKY & POPEO, 666 THIRD AVENUE, NEW YORK, NY, 10017, US

(21) Appl. No.: 10/440,932

(22) Filed: May 19, 2003

Related U.S. Application Data
(63) Continuation of application No. 09/624,184, filed on Jul. 25, 2000, now abandoned.

(30) Foreign Application Priority Data
Oct. 10, 1994 (FR) 94 12078

(51) Int. Cl. C12N 5/08

(52) U.S. Cl. 435/368

(57) ABSTRACT

The invention relates to optionally modified immortalized lines of endothelial brain cells of mammalians, as well as applications as preventive or curative drug and particularly for the treatment of primary and secondary, neurologic or psychiatric diseases, including brain tumor, and for stimulating the growth and reproduction of breeding animals. The invention also relates to the method for preparing said cell lines. The endothelial cell lines of mammalians disclosed are comprised of immortalized endothelial brain cells presenting at least one of the following characteristics of differentiated endothelial brain cells, in a stable way: the expression of endothelial markers, the secretion of vasoactive substances, the expression of molecules of the major histocompatilility complex (MHC), the expression of hormonal receptors, and the existence of tight junctions; said cell lines comprise a nucleic acid fragment having at least one immortalizing fragment of a viral or cellular oncogene, optionally associated with at least one selection gene, and an expression vector comprising a sequence coding for polypeptide, a protein or a viral vector, optionally associated with at least one selection gene and optionally at least one marker gene, and they are capable in vivo to integrate brain vessels of a host mammalian and produce said polypeptide, said protein or said viral vector.

The present invention relates to immortalized lines of mammalian endothelial brain cells, where appropriate modified, as well as to their applications as a medicinal product for preventive or curative use, and in particular for the treatment of various primary and secondary neurological or psychiatric disorders or diseases, including brain tumours.

For some years, new methods of treatment of a number of neurological disorders formerly considered to be refractory to all conventional treatments have made use of gene therapy. These new methods are linked, in particular, to the advances made in the field of the construction of effective expression vectors and of transporters of viral and cellular transgenes, and in the characterization of target cells suitable for gene therapy of the nervous system.

Two different approaches are essentially proposed for carrying out the transfer of genes into the nervous system: a so-called in vivo approach which focuses on the direct transfer of the genetic material to the cells in vivo, using viral and chemical agents, and an ex vivo approach which is characterized in that the gene transfer is performed in cells in culture, which are then implanted into the host body. The latter approach comprises steps of molecular manipulations, of cloning and of cell implantation so as to permit the distribution of the active substances in the host (SUHR S. T. et al., Arch. Neurol., 1993, 50, 1252-1268).

Many neurological disorders are associated with focused lesions or dysfunctions of the nervous system, and have hence been chosen to test these techniques.

The first trials in this field have essentially related to neurodegenerative diseases such as Alzheimer's disease or Parkinson's disease, and comprise the intracerebral grafting of foetal neural tissue or of adrenal medullary tissue in the brain (BJÖRKLUND A., TINS, 1991, 14, 8, 319-322).

The use of primary nervous tissues of foetal origin for cell transplantation in human therapy is a source of numerous ethical and practical problems; an alternative to this problem is to use primary cell lines of neural origin (for example neurons, glial cells, astrocytes) or non-neural cell lines (for example fibroblasts, myoblasts, chromaffin cells of the adrenal medulla, hepatocytes) (GAGE FH et al., Trends Neurosci., 1991, 14, 328-333). Although cell lines of adrenal medulla, of fibroblasts or of myoblasts can actually release active substances in vivo, they are not normally present in the nervous system, can modify the normal function of the nervous system and can give rise to a rejection reaction.

In point of fact, with the object of treating neurological or psychiatric disorders or diseases, including brain tumours, confined or otherwise to a specific region of the brain, it is necessary to have at one's disposal a cellular vector which is able to integrate completely in the nervous tissue while expressing a bioactive substance, in particular a protein, in a stable manner.

Consequently, it was the objective of the inventors to provide a cellular vector which meets better the needs encountered in practice, in particular in that it expresses, in a stable manner and in vivo, at least one previously selected polypeptide or protein or one viral vector, in that it is of brain origin, capable of integrating in the normal brain vascularization, and in that it is well tolerated.

The subject of the present invention is mammalian endothelial cell lines, characterized:

in that they consist of immortalized endothelial brain cells displaying at least one of the following features of differentiated endothelial brain cells, in a stable manner:

the expression of endothelial markers,

the secretion of vasoactive substances,

the expression of molecules of the major histocompatibility complex (MHC),

the expression of hormone receptors, and

the existence of tight junctions,

in that they comprise a fragment of nucleic acid comprising at least one immortalizing fragment of a viral or cellular oncogene, where appropriate in combination with at least one selectable gene, and an expression vector comprising a sequence coding for a polypeptide, a protein or a viral vector, where appropriate in combination with at least one selectable gene and where appropriate at least one reporter gene, and

in that they are capable in vivo of integrating in the brain vessels of a host mammal and of producing the said peptide, the said protein or the said viral vector.

For the purposes of the present invention, expression vector is understood to mean any nucleic acid fragment integrated in the genome or present in the cytoplasm, and capable of permitting the expression of the said polypeptide, protein or viral vector.

According to an advantageous embodiment of the said lines, the nucleic acid fragment comprising at least one immortalizing fragment of an oncogene contains the neomycin resistance gene and a fragment of the SV40 T oncogene.

According to another advantageous embodiment of the said lines, the nucleic acid fragment comprising at least one immortalizing fragment of an oncogene contains the E1A early region of the adenovirus 2 genome and the neomycin resistance gene.

According to another advantageous embodiment of the said lines, the said expression vector is a retroviral vector, in particular an MFG vector.

Preferably, the retroviral vector is an MFG-NB vector which is defective for replication.

The said vectors are described, in particular, in MULLIGAN et al. (Proc. Nat. Ac. Sci. USA, 1984, 81, 6349-6353) and FERRY et al. (Proc Natl Acad. Sci. USA, 1990, 88, 8377-8381).

Preferably also, the said endothelial cells are cells of brain capillaries.

Trials employing non-immortalized primary peripheral vascular endothelial cells have been described, but they do not constitute a suitable vector in that they do not constitute a pure, homogeneous and sufficient source for the purpose of a reproducible application to transplantation, and in that they do not display the endothelial brain phenotype.

According to yet another advantageous embodiment of the said cell lines, the sequence coding for a polypeptide or a protein is elected from the sequences coding for: enzymes such as proteases, enzyme inhibitors such as protease inhibitors, cytokines, neurotransmitters, neurotrophins, growth factors, toxins, antimetabolites, neurohormones, gangliosides, antibiotics, thrombolytic factors, coagulation factors, vasodilator or vasoconstrictor factors, hypo- or hypercholesterolaemic factors, hyper- or hypoglycaemic factors or any other substance of interest.

According to the invention, the said endothelial cells advantageously comprise, as immortalizing fragment, the E1A early region of the adenovirus 2 genome and the neomycin resistance gene, and, as vector, an MFG-NB retroviral vector containing the nls-lacZ gene coding for β-galactosidase. This cell line has been designated RBEZ by the inventors.

According to the invention, the said line has been deposited under the No. I-1481 dated Oct. 10, 1994 with the Collection Nationale de Cultures de Micro-organismes [National Collection of Microorganism Cultures] held by the Pasteur Institute.

Also according to the invention, the said endothelial cells advantageously comprise, as immortalizing fragment, the E1A early region of the adenovirus 2 genome and the neomycin resistance gene, and, as vector, a retroviral vector pMoMuLVisisNGF coding for murine β-NGF.

This cell line has been designated RBE/NGF-4 by the inventors.

According to the invention, the said line has been deposited under the No. I-1482 dated Oct. 10, 1994 with the Collection Nationale de Cultures de Micro-organismes held by the Pasteur Institute.

Unexpectedly, such endothelial cells of brain capillaries integrate well in the brain vascularization, are very well tolerated and release in vivo, over a long period, the active substance they express, and they find application in the preparation of a composition for the treatment of primary and secondary neurological or psychiatric disorders or diseases, including brain tumours, or for stimulating the growth and reproduction of livestock (poultry, sheep, cattle, pigs, horses, lagomorphs, rodents, and the like).

In particular, in the basal nucleus and in the striatum, many grafted endothelial cells according to the invention adopt a vascular localization. In both implantation sites, a not insignificant number of grafted cells are not associated with the host's vascular network. This non-vascular localization does not bring about an exacerbation of cell death, even at 1 year after implantation.

According to the invention, the viral vector is advantageously a modified cytomegalovirus (CMV) (integrative viral vector).

The subject of the present invention is also compositions, characterized in that they comprise at least one endothelial brain cell line according to the invention, in combination with at least one pharmaceutically acceptable vehicle.

Such compositions preferably contain between 10⁴ and 10⁵ endothelial cells/μl.

Such compositions may be advantageously administered via the intracranial, subcutaneous, intracerebroventricular, subdural, venous or arterial (for example intracarotid), intramuscular or intrathecal route.

According to the invention, the said endothelial cells may be cells of the same species as the host (allograft or homograft) or of a different species (xenograft).

The subject of the present invention is also a method for obtaining a modified cell line according to the invention, which method is characterized in that:

(1) a first transfection is carried out by:

culturing endothelial brain cells, preferably those of brain microvessels, in a suitable culture medium supplemented with serum and with growth factors,

transfection of the said cells between the 2nd and the 6th passage with a nucleic acid fragment comprising at least one immortalizing fragment of a viral or cellular oncogene and, where appropriate, at least one selectable gene, in particular a gene coding for resistance to an antibiotic,

selection of the transfected cells on a selection medium suited to the said selectable gene, if necessary,

(2) a transfection of the cells obtained in (1) is then carried out with a vector containing the polypeptide sequence or protein sequence to be produced or a viral vector to be expressed.

Preferably, the transfection of step (2) is carried out with a retroviral vector in which the sequence coding for the protein to be expressed has been incorporated beforehand.

Preferably, step (1) enables RBE4 cells to be obtained, which cells are immortalized by transfection with a plasmid containing the E1A early region of the adenovirus 2 genome and the neomycin resistance gene under the control of the SV40 promoter, and which are deposited under the No. I-1142 with the Collection National de Micro-organismes (CNCM) held by the Pasteur Institute.

The subject of the present invention is also a model for studying the integration in the brain of cells that deliver active substances to the brain, characterized in that it comprises an REBZ cell line according to the invention.

The subject of the invention is also a model for studying and identifying the biochemical and cellular systems of the blood-brain barrier in vitro, characterized in that it comprises at least one cell line according to the invention.

The subject of the present invention is, in addition, a method for producing a polypeptide or a protein, characterized in that it comprises the use of at least one endothelial cell line according to the invention, in a suitable bioreactor.

Besides the foregoing arrangements, the invention also comprises other arrangements which will become apparent from the description which follows, which relates to examples of implementation of the method which is the subject of the present invention as well as to the attached drawings, wherein:

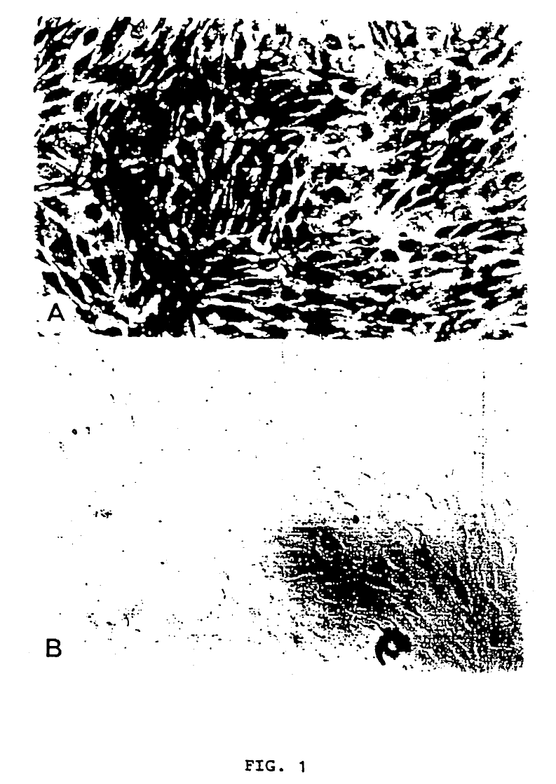

FIG. 1 illustrates the in vitro study of the expression of the NGF transgene in RBE/NGF cells, by in situ hybridization;

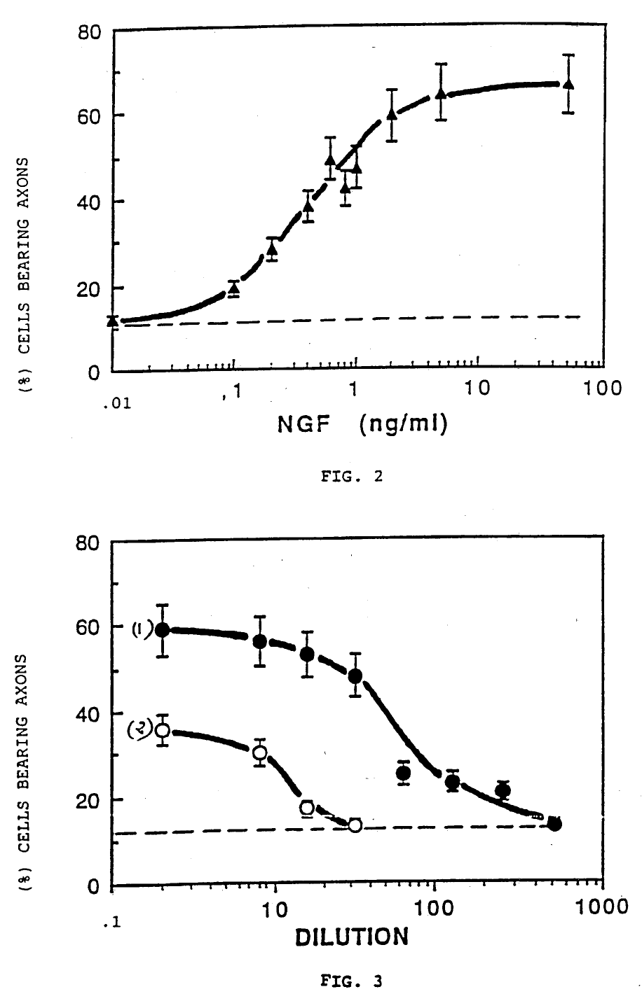

FIGS. 2 and 3 illustrate the stimulation of axonal budding of PC12 cells, obtained from the supernatant of RBE/NGF cells in vitro;

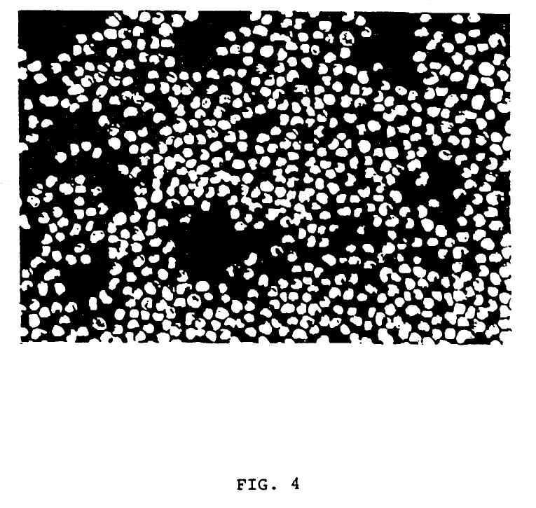

FIG. 4 illustrates the prelabelling of RBE4 cells before transplantation, with the nuclear stain Hoechst 33342 (bisbenzimide);

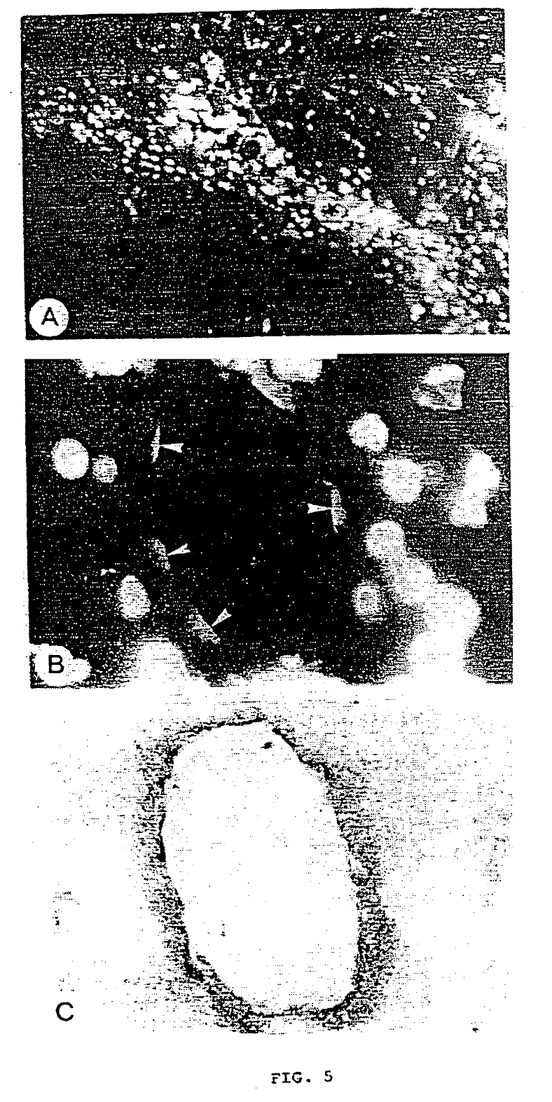

FIG. 5 illustrates the visualization of the cells prelabelled with the Hoechst stain, after transplantation into adult rat brain;

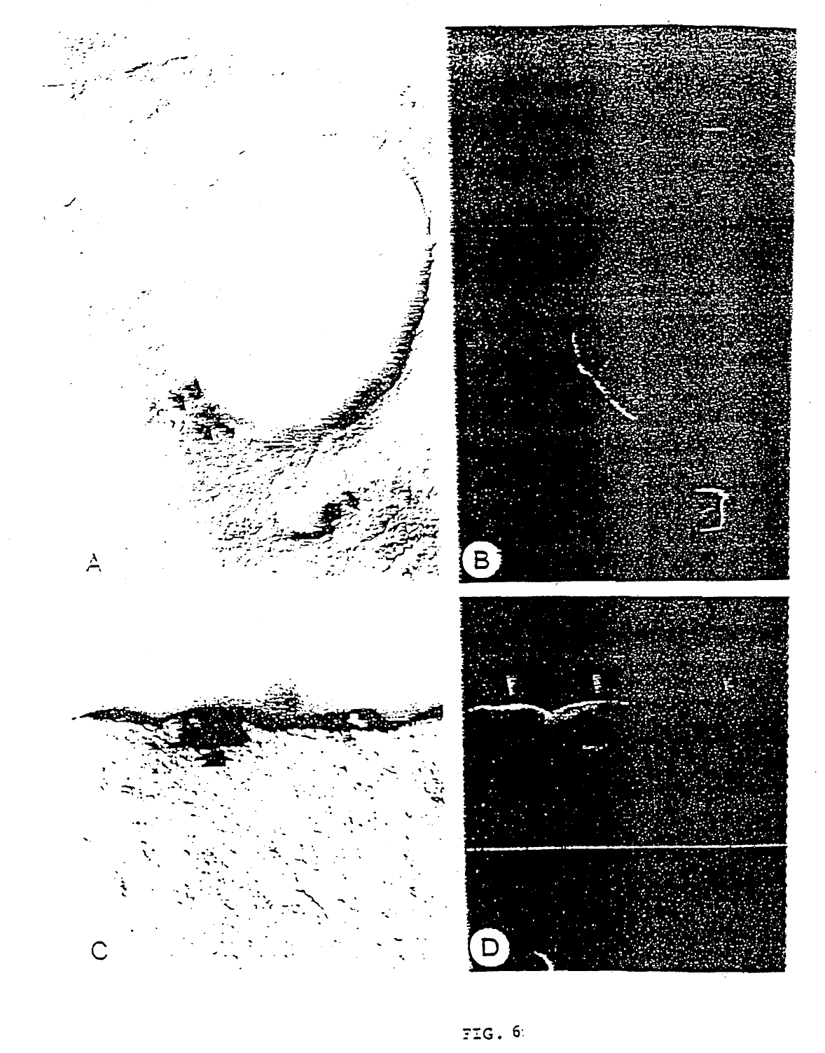

FIG. 6 illustrates the study of the morphological and functional integration of RBEZ cells, by visualization of the expression of the nls-lacZ transgene and of the antigenic marker of integrity of the blood-brain barrier (BBB), EBA (endothelial barrier antigen);

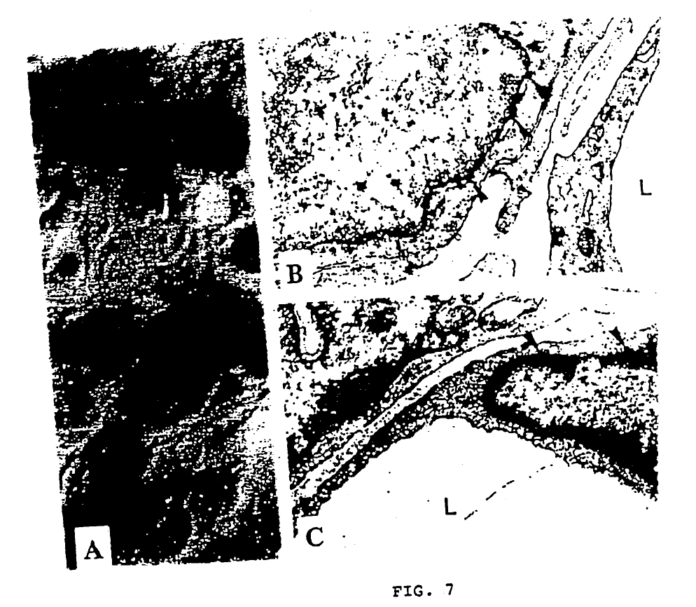

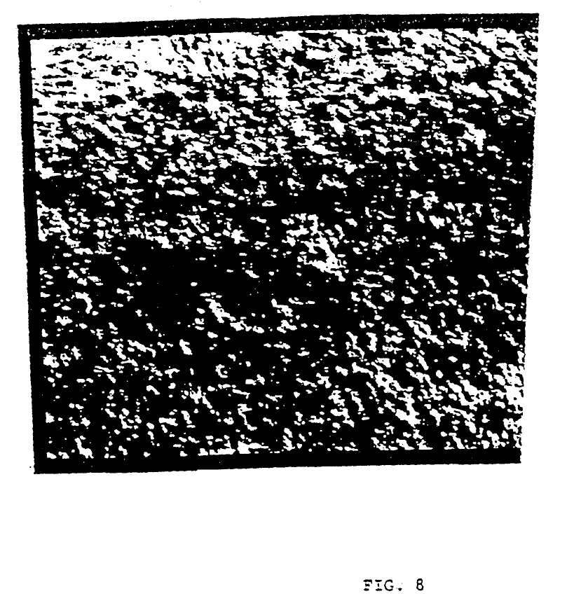

FIG. 7 illustrates the ultrastructural study by electron microscopy, demonstrating the morphological and functional integration of RBEZ cells after intracerebral grafting, by visualization of the expression of the nls-lacZ transgene;

FIG. 8 illustrates the study of the morphological and functional integration of RBEZ cells after grafting into an intracerebral tumour, by visualization of the expression of the nls-lacZ transgene;

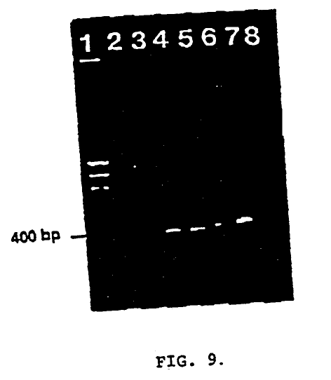

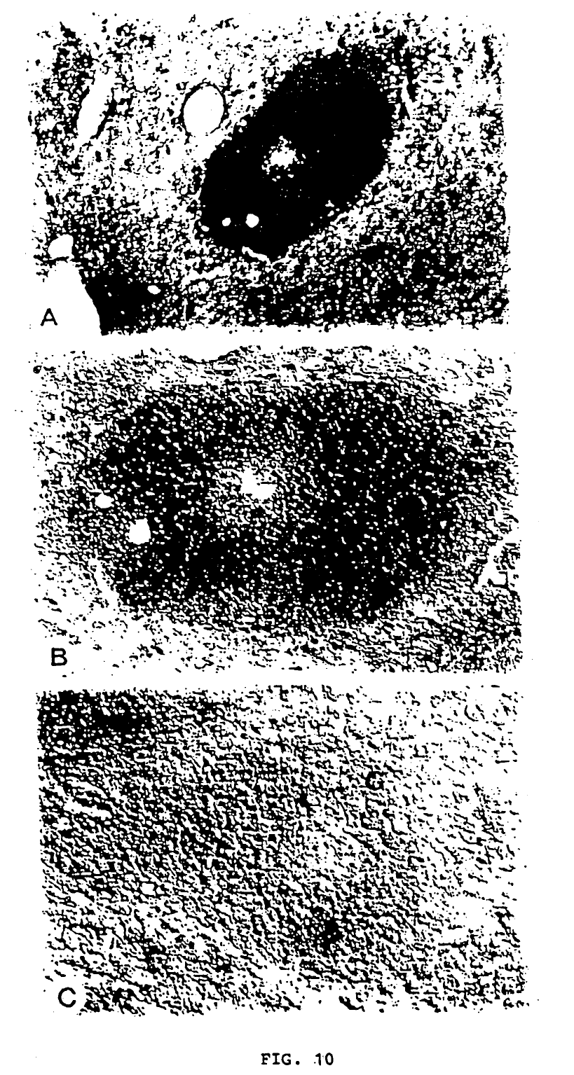

FIG. 9 illustrates the identification of the nls-lacZ gene in tumours implanted with RBEZ cells, by PCR.

FIG. 10 illustrates the in vivo study of the expression of the NGF transgene in RBE/NGF cells, three weeks after transplantation into the nucleus basalis (basal nucleus);

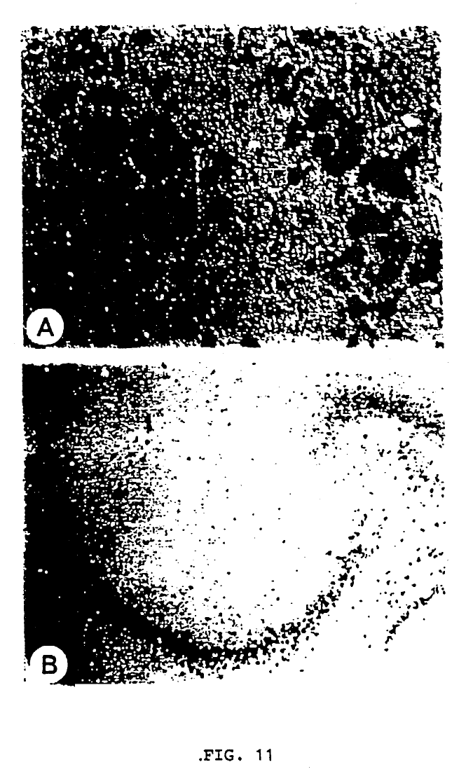

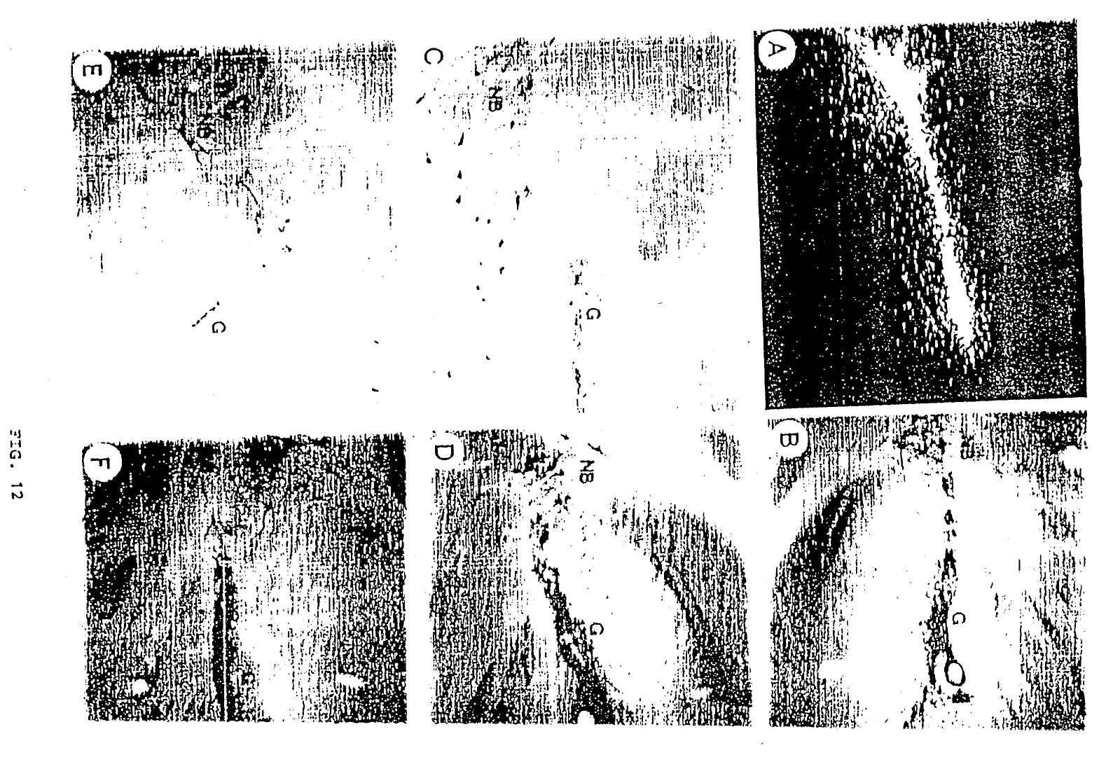

FIG. 11 illustrates the control brain structures used as internal control of the in situ hybridization of the NGF messenger, in vivo;

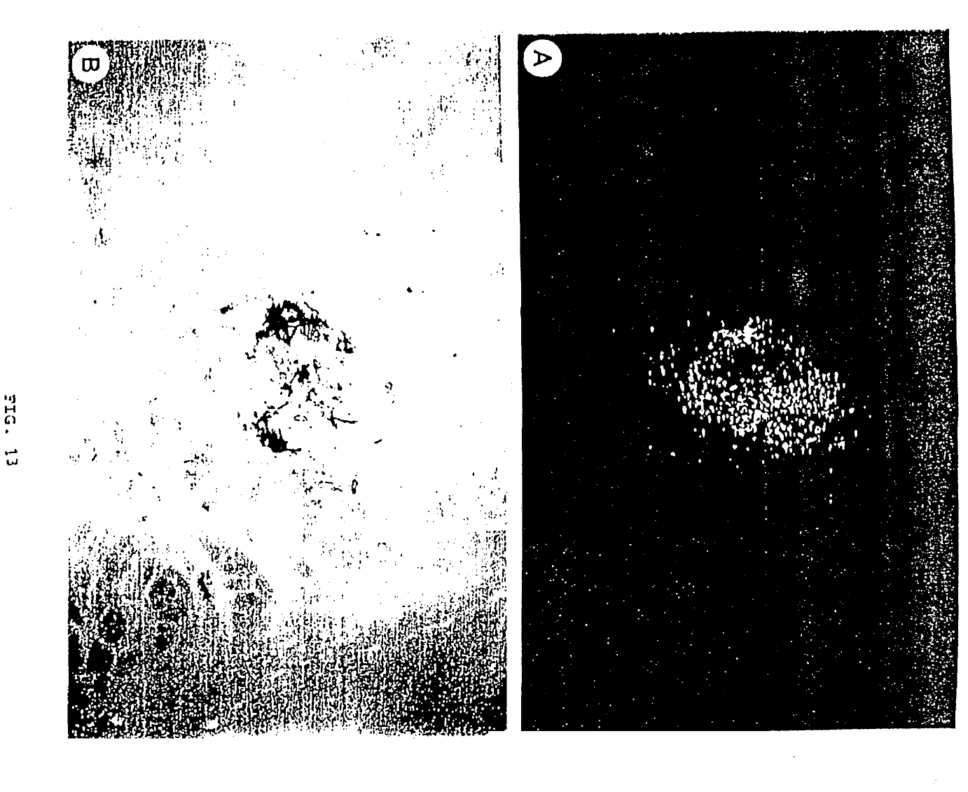

FIG. 12 illustrates the biological effect of the NGF secreted by RBE/NGF cells, three weeks after grafting, in the nucleus basalis;

FIG. 13 Illustrates the biological effect of the NGF secreted by RBE/NGF cells, three weeks after grafting, away from the nucleus basalis;

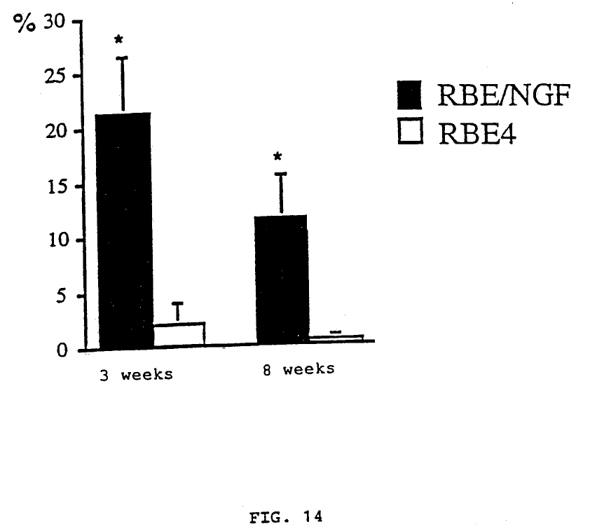

FIG. 14 illustrates the quantification of the biological effect induced by the expression of the NGF transgene at 3 and 8 weeks after grafting, and this is reflected in he area occupied by the p75LNGFR immunolabelling relative to the area of the graft.

It should, nevertheless, be clearly understood that these examples are given only by way of illustration of the subject of the invention, and in no way constitute a limitation of the latter.

### EXAMPLE 1

Preparation of RBE4 Immortalized Endothelial Brain Cells.

Endothelial cells of microvessels of rat brains (primary culture) are immortalized by transfection with the plasmid pE1A-neo, containing the sequence coding for the adenovirus E1A followed by the neomycin resistance gene.

A clone designated RBE4 was obtained in this way, and its features are described, in particular, in Application PCT WO 93/06222 as well as in the papers published in J. Cell. Physiol., 1993, 155, 104-111 and J. Cell. Physiol., 1994, 159, 101-113. This clone was deposited under the No. I-1142 with the Collection Nationale de Cultures de Micro-organismes (CNCM).

To carry out the transfection, the calcium phosphate coprecipitation technique was used, as described in Application PCT WO 93/06222 and repeated below: the transfection of the said cells is carried out at the 5th passage with the abovementioned plasmid (10 μg) containing, besides the E1A early region of the adenovirus 2 genome and the neomycin resistance gene, the SV40 promoter.

This transfection takes place after culturing these cells in collagen-coated dishes containing an α-MEM/F10 (2/3; 1/3) medium supplemented with 10% foetal calf serum (FCS), 1 ng/ml FGFb, glutamine (2 mM) and penicillin/streptomycin. The cell line obtained possesses some of the features of endothelial brain cells; it possesses, in particular, an untransformed phenotype: contact inhibition, growth factor- and adhesion factor-dependent proliferation, expression of endothelial differentiation markers (antigen related to factor VIII), binding site for Griffonia simplicifolia agglutinin and absence of tumorigenic effect in nude mice.

Furthermore, these cells are stimulated by astrocytes to express the specific enzymatic markers of the blood-brain barrier, namely γ-glutamine transferase and alkaline phosphatase.

### EXAMPLE 2

Preparation of RBEZ Endothelial Brain Cells.

The RBE4 cells obtained in Example 1 are subjected to two passages per week on an α-MEM/F10 (1/1; Seromed, France) medium supplemented with 2 mM glutamine, 10% foetal calf serum, 1 ng/ml FGFb and 300 μg/ml G418.

They are plated out at a density of 10⁴ cells/cm² on collagen-coated dishes, and used between passages 30 and 60.

1) Preparation of the Retroviral Vector:

An MGF-NB vector which is defective for replication and contains the lacZ gene is obtained by inserting the sequence coding for E. coli β-galactosidase fuzed to a sequence coding for the nuclear localization sequence (nls) of 21 amino acids originating from the SV40 T antigen (Kalderon D. et al., Cell, 1984, 39, 499-509). This vector, MFG-NB nls-lacZ, is introduced into ψ-2 retrovirus-producing cells (Mulligan et al., loc. cit.) (recombinant retroviral infection of ψ-2) and enables ψ-2-MFG-NB cell lines to be obtained.

These retrovirus-producing cells are plated out in dishes at a density of 10⁶ cells per dish 10 mm in diameter in 7 ml of RPMI 1640 medium supplemented with 10% foetal calf serum.

After 24 h, a volume of 6 ml of medium containing the virus is filtered and used for infection, or alternatively stored at 80° C. until used.

2) Infection of RBE4 Endothelial Cells:

RBE4 cells are plated out on dishes at a density of 10⁴ cells/cm² and, after 24 h, the virus (3 ml) is added in the presence of polybrene (10 μg/ml) for 2 h.

After a further 24 h period in complete medium, the RBE4 cells are subcultured and reinfected under the same conditions.

3) Selection of Endothelial Cells Expressing the Transgene:

The cells expressing β-galactosidase (RBEZ cells) are sorted by FACS (fluorescent activated cell sorting; NOLAN et al., PNAS, 1988, 85, 2603-2607) using fluorescein β-D-galactopyranoside (FDG) as substrate for the enzyme.

According to this technique, 10⁶ RBEZ cells in 100 μl are incubated at 37° C. for 5 min in a 5 ml polystyrene tube before adding 100 μl of FDG (2 mM).

After mixing, the cells are placed again at 37° C. for 1 min and then on ice, and the volume is adjusted to 2 ml.

4) Detection of the Expression of the Transgene by Visualization of the β-Galactosidase Enzyme Activity using a Chromogenic Substrate, X-Gal:

Protocol:

The enzyme activity is detected by incubating the cells at 37° C. in PBS buffer containing 2 mM 5-bromo-4-chloro-3-indolyl β-D-galactopyranoside (X-gal), 20 mM potassium ferricyanide, 20 mM potassium ferrocyanide and 2 mM MgCl₂.

Results:

The presence of a β-galactosidase enzyme activity is revealed by the formation of a blue coloration. Approximately 50-80% of RBE4 endothelial cells infected with the retrovirus stain blue in the histochemical situation, but the level of coloration varies from one cell to another Control (uninfected) RBE4 cells are not stained under the same conditions.

5) Properties of RBEZ Cells in Vitro:

The RBEZ cells obtained are cultured on a collagen-coated support in an α-MEM/F10 medium supplemented with 10% foetal calf serum, 2 mM glutamine, 1 ng/ml FGFb and 300 μg/ml G418.

These cells display contact inhibition and growth factor- and adhesion factor-dependent proliferation; they express, in addition, endothelial differentiation markers.

### EXAMPLE 3

Preparation of RBE/NGF Endothelial Brain Cells.

The RBE4 cells obtained in Example 1 are subjected to two passages per week on an α-MEM/F10 (1/1; Seromed, France) medium supplemented with 2 mM glutamine, 10% foetal calf serum, 1 ng/ml FGFb and 300 μg/ml G418.

They are plated out at a density of 10⁴ cells/cm² on collagen-coated dishes and used between passages 30 and 60.

1) Preparation of the Retroviral Vector:

The procedure is as in Example 2, using a retroviral vector pMoMuLVisisNGF which is deficient for replication and into which the sequence coding for mouse β-NGF is inserted (Scott J et al., Nature, 1983, 302, 538-540). This vector, introduced into ψ-2 producing cells, enables ψ-2-MoMuLVisisNGF cell lines to be obtained.

2) Infection of RBE4 Endothelial Cells:

The procedure is as in Example 2.

3) Selection of Endothelial Cells Expressing the Transgene by a Two-Site ELISA Method:

Following subcloning of the infected RBE4 cells by the limiting dilution method, subclones secreting βNGF (RBE/NGF cells) are identified using a two-site ELISA (LADENHEIM et al., J. Neurochem., 1993, 60, 260-266).

More specifically, an anti-βNGF monoclonal antibody designated 27/21 (0.1 mg/ml in 0.05 M carbonate buffer pH 9.6) is applied to Costar EIA/RIA plates for 2 hours at 37° C. The plates are washed 3 times with a mixture of 50 mM Tris-HCl, pH 7.4, 200 mM NaCl, 10 mM CaCl₂, 0.1% Triton X-100 and 0.05% sodium azide, and incubated at 4° C. overnight in a conditioned medium or βNGF standards (30-1000 pg/ml) in the same buffer supplemented with 1% bovine serum albumin.

After washes, βNGF is detected using the same antibody conjugated to β-D-galactosidase (400 mU/ml) after incubation for 4 hours at 37° C. The chromogenic substrate is chlorophenol red β-galactopyranoside (1 mg/ml in a 100 mM Hepes, pH 7, 150 mM NaCl, 2 mM MgCl₂, 0.1% sodium azide medium). The absorbance at 570 nm is read after 2 hours at 37° C. Two highly positive subclones designated RBE/NGF-2 and -4 are selected, as well as two less positive subclones, from 94 clones tested.

4) Cellular Detection of NGF Synthesis by in situ Hybridization of the Nucleotide Sequence (mRNA) Coding for NGF:

An in situ hybridization is carried out with a 48-mer antisense probe specific for the nucleotide sequence (mRNA) coding for βNGF, corresponding to positions 897-944 of the cDNA sequence of mouse βNGF (SCOTT et al., 1983, Nature 302, 538-540), of the following formula:

48-mer mature NGF 5′−3′ antisense sequence

5′ CTG CTT CTC ATC TGT TGT CAA CGC CTT GAC GAA GGT GTG AGT CGT GGT 3′,

so as to visualize the βNGF transcript in the infected cells in culture. These results show a substantial expression of βNGF mRNA in the infected cells, at a level which is variable from one cell to another.

FIG. 1 illustrates the in vitro study of the expression of the NGF transgene in RBE/NGF cells, by in situ hybridization.

The immunoenzymatic visualization of the expression of the NGF transgene is carried out using a digoxigenin-labelled antisense oligonucleotide probe specific for murine NGF. Under these conditions, the mRNA/NGF probe hybrids are visualized with an anti-digoxigenin antibody coupled to alkaline phosphatase, the enzymatic reaction of which with the NBT-BCIP substrate complex produces a blackish precipitate. FIG. 1A shows an intense signal in the RBE/NGF cells in culture, indicating a high level of expression of the NGF transgene. In 1B, the absence of a positive reaction in uninfected control RBE4 cells is observed (×300 in 1A and 1B) (1B in phase contrast).

5) In Vitro Activity of the Secreted NGF:

The biological activity of the NGF secreted into the supernatant of the RBE/NGF cells is demonstrated by the property of promoting a budding of axons from rat phaeochromocytoma PC12 cells. To carry out this test, the RBE/NGF cells are plated out on dishes at a density of 10⁴/cm² in dishes 100 mm in diameter, and growth is carried out for 3 to 4 days to confluence (10⁷ cells/dish). The medium is changed (10 ml) and the supernatants are collected after 24 hours. The results are illustrated in FIGS. 2 and 3, which show that the 24-hour cell supernatant behaves in the same manner as purified NGF (0.1-50 ng/ml) used as internal standard, and leads to a stimulation of axonal budding in approximately 65% of the PC12 cells. In addition, a 1:40 dilution of the said supernatant displays a biological activity comparable to NGF at a concentration of 0.4 ng/ml (40% of cells bearing axons). Consequently, the capacity for secretion of biologically active NGF by the RBE/NGF cells may be estimated at 16 ng/10⁶ cells/24 hours.

These FIGS. 2 and 3 show, as abscissae, the NGF concentration (ng/ml) (FIG. 2) or the degree of dilution (FIG. 3; curve 1: RBE/NGF cells, and curve 2: RBE4 cells), and as ordinates, the percentage of cells bearing axons.

### EXAMPLE 4

Implantation in the Brain of RBE4, RBEZ and RBE-NGF cells.

I. RBE4 Cells: Survival and Integration.

For the characterization of the RBE4 cell lines transplanted into adult rat brain, a method of prelabelling with Hoechst bisbenzimide is used in order to monitor the grafted cells (GANSMÜLLER et al., GLIA, 1991, 4, 580-590).

FIG. 4 illustrates the prelabelling of RBE4 cells before transplantation, with the nuclear stain Hoechst 33342 (bisbenzimide). The suspended cells are visualized in fluorescence microscopy under ultraviolet light. The fluorescence of the stain clearly defines the positively labelled cell nuclei (×270).

Three to eight weeks after implantation of labelled RBE4 cells in different regions of the brain (grey matter and white matter), the graft has a compact appearance with a small and constant spread of some RBE4 cells around its mass. This migration takes place essentially along the host's vascular network, suggesting a preferential interaction between the implanted endothelial cells and the host's vascular components.

Histological staining of the brains grafted in this way show a minimum of signs of necrotic cells, this occurring essentially during the first week following the surgical trauma due to the transplantation.

Within the graft, the cell density is homogeneous (little or no presence of pyknotic cells). The GFAP (glial fibrillary acidic protein) immunoreactivity characteristic of astrocytes is considerable from the first week after implantation, both around the graft and in the graft itself, indicating an infiltration of astrocytes into the latter.

Unexpectedly, the implanted RBE4 cells migrate and integrate in the vascular environment, sometimes with a direct participation in the host's vascular network.

FIGS. 5A, 5B and 5C show the cells prelabelled with the Hoechst stain, after transplantation into adult rat brain. FIG. 5A shows a general view of the region of grafting in the brain parenchyma. The fluorescent grafted endothelial cells appear to accumulate preferentially around vascular components of the host's brain. The asterisks symbolize the course of a blood vessel (×250). FIGS. 5B and 5C show a blood vessel at high magnification, located in the region of implantation of the graft. In 5B, numerous Hoechst-positive RBE4 cells integrated in a luminal (arrows) and perivascular position may be observed. In 5C, this same vessel is immunolabelled with an anti-laminin (specific marker of blood vessels) antibody (×600).

II. RBEZ cells: Survival, Integration and Expression of the Transgene.

1) Prelabelling of Cells with Hoechst Bisbenzimide.

See I.

2) Preparation of Cells Before Their Implantation.

Immediately before the grafting procedure, the cells are rinsed three times in a grafting solution comprising PBS supplemented with MgCl₂ and CaCl₂ at a concentration of 1 μg/ml and glucose at a concentration of 0.1%, so as to remove the DMEM-FCS medium.

3) Surgery and Implantation of Cells.

Adult male rats belonging to the Lewis strain and weighing 300 g receive a graft of prelabelled RBEZ cells, as specified in 1), under deep anaesthesia, under stereotactic conditions (Kopf® stereotactic frame, Paxinos atlas of the rat's brain) Twenty animals receive, respectively, a stereotactic implantation in the basal nucleus of RBEZ cells (right cerebral hemisphere) and of control RBE4 cells (left cerebral hemisphere).

A total of 300,000 cells suspended in a grafting solution (3 μl) is injected per site using an Exmire® 10 μmicrosyringe having an external needle diameter of 0.5 mm.

4) X-gal histochemical visualization for light microscopy.

The anaesthetized rats are sacrificed by perfusion with 150 ml of PBS and then with 300 ml of 4% PFA in a PBS solution (0.1 M, pH 7.4) at 4° C.

To visualize the presence of β-galactosidase, the brains are cryoprotected and frozen by inclusion in a compound OCT® or cryostat sectioning. After sectioning, the enzyme activity of the nls-lacZ transgene is detected by incubating the tissue at 37° C. in PBS buffer containing 2 mM 5-bromo-4-chloro-3-indolyl 62 -D-galactoside (X-gal, Sigma), potassium ferricyanide (20 mM), potassium ferrocyanide (20 mM) and MgCl₂ (2 mM). These reaction conditions do not give rise to staining in control animals and for the grafted unmodified RBE4 cells (cells grafted on the left-hand side). The cellular localization and the morphology in the tissue sections is increased if necessary using Nomarski optics, which permits a reliable identification of the cell type in the vascular structure. This visualization is combined, if necessary, with EBA immunohistochemistry for a cell characterization in greater depth.

FIG. 6 illustrates the study of the morphological and functional integration of RBEZ cells, by visualization of the expression of the nls-lacZ transgene and of the antigenic marker of integrity of the blood-brain barrier (BBB), EBA (endothelial barrier antigen). FIGS. 6A, 6B, 6C and 6D show blood vessels located away from the region of grafting, onto which RBEZ cells have migrated after transplantation. In these figures, nuclei of endothelial cells, expressing the nls-lacZ transgene, in a luminal position (arrows) (6A, 6C), may be observed. These same vessels, in fluorescence illumination (6B, 6D), express The EBA antigen (arrow heads), thereby indicatlng that the vascular insertion of the grafted cells has not impaired the integrity of the BBB (×750 in 6A and 6B); (×1500 in 6C and 6D); (6A and 6C, in transmitted interference contrast). Calculation of the percentage of grafted cells expressing the transgene was undertaken. Thus, at one week after grafting, 6.9±0.6% of the grafted Hoechst-positive cells express β-galactosidase.

Expression of the transgene is considerable up to 5 weeks after implantation, but decreases afterwards. The presence of implanted cells remains, however, detectable using the abovementioned Hoechst stain. The absence of specific major changes in the host's immune reaction with respect to the presence of the lacZ transgene shows that these RBEZ cells are properly integrated.

In addition, the RBE4 and RBEZ cells never develop tumours in vivo, since they display a great stability of their phenotype.

5) X-gal histochemical visualization for electron microscopy.

Animals (n=10) were treated for an ultrastructural study of the integration of the RBEZ cells by electron microscopy. In this case, the animals were perfused with PBS solution containing 2.5% of PFA and 0.5% of glutaraldehyde. The brains are removed and left in the same fixative overnight. After rinsing, they are cut with a vibratome into sections of thickness 75 μm.

For the visualization of cells expressing the lacZ gene, the substrate X-gal was used as for light microscopy, which, under the action of β-galactosidase, forms a precipitate which is dense to electrons and visible in the electron microscope.

A prelocalization of the graft is performed on sections before treatment with 1% OsO₄. These thick sections are thereafter dehydrated and then included in Epon.

The tissue blocks are thereafter sectioned with an ultramicrotome into semithin and ultrathin sections, which are counterstained or otherwise with uranyl acetate and lead citrate and then examined on a JEOL CX100 instrument.

FIG. 7 illustrates the study of the morphological and functional integration of RBEZ cells by visualization of the expression of the nls-lacZ transgene by electron microscopy. FIG. 7A shows, in Nomarski optics, the perinuclear β-galactosidase labelling in the grafted cells on a semithin section of brain (2 μm) (×1660). On examination in electron microscopy, the cells are observed either in the parenchyma (FIG. 7B) or in a vascular position (FIG. 7C), forming blood vessels of the host. The arrow heads point to the perinuclear precipitates of X-gal, which are dense to electrons. Even integrated in the brain parenchyma, in the absence of direct contacts with the blood compartment, these endothelial brain cells are capable of surviving for long periods. The grafted cells appear to be metabolically active and capable of establishing specialized connections between themselves and with the cells of the host (presence of desmosomes and of tight junctions). In the vascular position, the RBEZ cells display a normal phenotype from the first week after grafting (tight junction and few pinocytotic vesicles) (×60,000 in 7B and 7C) (L: vascular lumen).

III. RBEZ Cells and Brain Tumours

1) Implantation:

RBEZ cells at confluence are trypsinized and resuspended in DMEM without serum immediately before they are implanted in the host animals.

For an Intracranial Implantation, the RBEZ cells (5×10⁵ cells) are injected stereotactically with a syringe (Hamilton, gauge 26, with a bevelled end), into the caudate nucleus and the putamen of Fischer 344 rats (200 to 250 g) after anaesthesia.

The cells are injected in a volume of 5 μl and the needle is left in place for 2 min after injection in order to limit leakage.

In the Context of a Subcutaneous Implantation, anaesthetized Fischer rats receive 100 μl containing 10⁶ RBEZ cells.

To show that such cells implant preferentially in hypervascularized regions such as tumours, a trial is carried out performing the same implantations (intracranial and subcutaneous) with a mixture of F98, C6 or 9L glioma cells (10⁵ cells) and RBEZ cells, under the same conditions as above.

After implantation, the tissues are prepared so as to perform an immunohistochemical and histological study.

2) Preparation of Tissues:

Rats are anaesthetized with ether and, after thoracotomy, the right atrium is incised and a cannula is inserted into the left ventricle, which is then perfused. sequentially with a buffer containing 120 mM NaCl, 2.7 mM KCl in phosphate buffer pH 7.4 (1 ml/g of weight) and then 3.7% paraformaldehyde (fixative). The brains are placed in the same fixative for 30 minutes, cryoprotected with 30% sucrose in PBS and frozen. The tissues are cut (thickness 12 μm) and mounted on gelatin-coated slides.

3) Demonstration of the Survival of the Cells and of the Expression of the Transgene:

a) Histochemical and Immunohistochemical Protocol for the Detection of RBEZ Cells Expressing the nls-lacZ Reporter Gene:

. Histochemical Study

The slide preparations are rinsed three times in PBS buffer and then Incubated at 37° C. for 1 to 2 hours in PBS containing 0.5 mg/ml of 5-bromo-4-chloro-3-indolyl β-D-galactopyranoside (X-gal), 20 mM potassium ferricyanide, 20 mM potassium ferrocyanide and 2 mM MgCl₂. Sections incubated in the absence of X-gal substrate are used as a negative control. The sections are then rinsed in PBS buffer and mounted in 90% glycerol in PBS containing 0.02% of sodium azide.

The reaction conditions do not give rise to any staining in control animals which have not received any RBEZ cell.

. Immunohistochemical Study

A few sections display a positive reaction for laminin in the tumour (detection of tumour microvessels), for the nuclear proliferation antigen Ki67 and for the expression of markers of the blood-brain barrier, such as the endothelial glucose transporter type 1 (glut-1), after staining with X-gal. For staining laminin, the sections are digested for 15 minutes at 37° C. with 0.2% pepsin in 0.01 N HCl before incubation with the immunological reagents.

The sections are incubated sequentially with 1% normal goat serum and then with either rabbit anti-laminin antibodies, rabbit anti-glut-1 antibodies or rabbit anti-Ki67 antibodies, and biotinylated goat anti-rabbit immunoglobulin is then added (1:200 in PBS).

The sections are then incubated with an avidin-biotin-peroxidase complex (1:50 in PBS) followed by an incubation in 50 mM Tris buffer containing 0.5 mg/ml of 3,3′-diaminobenzidine (Sigma) and 0.01% of hydrogen peroxide.

Control slides are incubated with normal rabbit serum in place of the immune serum. The sections are mounted in 90% glycerol in PBS.

b) Results

1) Visualization of Implanted RBEZ Cells

After staining with the chromogenic substrate X-gal, the blue product of the reaction with β-galactosidase is identified in histological sections of the intracranial and subcutaneous tumours after grafting of tumour and RBEZ cells (Tables I and II).

No blue reaction product is detected in the tumours implanted without RBEZ cells. Surprisingly, the grafted endothelial cells are distributed throughout all the intracranial tumours including the marginal infiltrations, but do not appear to migrate into the normal tissues.

2) Integration of Implanted RBEZ Cells

Essentially all the β-galactosidase-positive cells, irrespective of their localization, stain with the anti-laminin antibodies, this being in agreement with their endothelial phenotype. Interestingly, a few grafted RBEZ cells are associated with tumoral microvascular profiles.

FIG. 8 illustrates the integration of RBEZ cells in the tumour tissue (C6 cells) and its vascular network (arrow head) on a section of brain tissue counterstained with neutral red and in Nomarski optics.

This suggests that RBEZ cells implanted in this way have the capacity to integrate in an anatomically correct manner in the tumour vascularization.

3) Functionalities of Integrated RBEZ Cells

One of the most important features of these endothelial brain cells is their high expression of the glucose transporter type 1 (glut-1), expressed at the blood-brain barrier. This is important for the transendothelial transport of D-glucose, the essential energy-releasing substrate of the brain. The endothelial cells in 9L or other intracranial gliomas also express this transporter. In contrast, the expression of glut-1 decreases rapidly in endothelial brain cells in culture.

Many β-gal-positive cells are labelled with the anti-glut-1 antibodies. These different tests show that endothelial brain cells genetically modified ex vivo survive, integrate in intracranial gliomas and express the transgene.

4) Proliferation of Implanted RBEZ Cells

The expression of the Ki67 proliferation antigen by RBEZ cells implanted in intracranial 9L tumours is examined.

Many cells expressing both nuclear β-galactosidase and Ki67 antigen are observed.

The number of RBEZ cells implanted per tumour section is quantified by computer-assisted image analysis using the imaging device (MCID) supplied by the company Imagine Research Inc. (Brock, St Catherine's University, Ontario, Canada), a Hamamatsu high-resolution CCD camera and a Compaq DeskPro 486/33 computer.

The total number of RBEZ cells per tumour is estimated from the number of RBEZ cells per tumour volume (12 μm adjacent section), and the total tumour volume is estimated from the limits of the tumour according to two orthogonal planes of sectioning.

Tables I and II illustrate the results obtained on implantation of the modified endothelial brain cells according to the invention in 9L gliomas.

TABLE I

| Number of | | Number of days |
|---|---|---|
| tumours | Number of β-Gal + | after implantation |
| examined | tumours | of RBEZ cells |
| 4 | 4 | 7 |
| 10 | 10 | 12 |
| 4 | 4 | 14 |
| 3 | 3 | 21 |
| 3 | 3 | 28 |
| 4 | 4 | 35 |

TABLE II

| | | Number of RBEZ |
|---|---|---|
| | | cells/ |
| | Tumour type | tumour |
| | (days) | (mean ± SEM) |
| Intracranial | 9L (D12) | 166,440 ± 19,550 |
| | C6 (D12) | 145,840 ± 42,160 |
| | F98 (D12) | 232,560 ± 69,070 |
| Subcutaneous | 9L (D14) | 494,560 ± 422,500 |
| | 9L (D21) | 5,252,160 ± 611,380 |

5) Identification of the nls-lacZ Gene in Tumours with RBEZ Cells, by PCR

Oligonucleotides complementary to DNA sequences localized on the nls-lacZ gene (5′-CGACTCCTGGAGCCCGTCAGTATC-3′) and on the vector, downstream of the 3′LTR sequence (5′-GACCACTGATATCCTGTCTTTAAC-3′), are used as primers. PCR is carried out on genomic DNA isolated from control tumours (9L tumours; FIG. 9, lanes 2 and 3), from experimental tumours (tumours which have integrated RBEZ cells: implantation 14 days before isolation of the DNA; FIG. 9, lanes 4-6) and from the RBEZ (FIG. 9, lane 7) and RBE4 (FIG. 9, lane 8) cell lines. 35 amplification cycles with Taq polymerase are carried out under the following conditions: denaturation at 95° C., hybridization at 60° C. and elongation at 72° C.

FIG. 9 shows the results obtained: the PCR product (400 bp) is present only in the samples containing RBEZ cells.

IV. RBE/NGF Cells.

1) Prelabelling with Hoechst Bisbenzimide, See I.

2) Implantation:

Immediately before the grafting procedure, the cells are rinsed three times in a grafting solution comprising PBS supplemented with MgCl₂ and CaCl₂ at a concentration of 1 μg/ml and with glucose at a concentration of 0.1%, so as to remove the DMEM-FCS medium.

Surgery and Implantation of Cells:

A total of 50 adult male rats divided into 2 groups, belonging to the Lewis stain and weighing approximately 300 g, receive a graft of RBE/NGF cells prelabelled with the Hoechst stain, under deep anaesthesia, under stereotactic conditions.

Ten animals receive a stereotactic implantation of RBE/NGF cells in the right basal nucleus. Another group (n=40) is subjected to a procedure of injections at multiple sites of RBE/NGF cells (right-hand side) so as to produce a cell column 2 mm in height between the basal nucleus and the dorsal striatum. A total of 300,000 cells suspended in a grafting solution (3 μl) is injected per site using an Exmire® 10 μl microsyringe with an external needle diameter of 0.5 mm.

As a control procedure, unmodified RBE4 cells labelled with the Hoechst stain are also grafted contralaterally (left-hand side) at the same time and using the same stereotactic levels. Coronal and horizontal sections of the grafted brains are collected between 1 week and 12 months after transplantation. The grafts examined, visualized by the fluorescence obtained using the Hoechst stain, show a compact appearance with little cellular spreading. No tumorigenic effect on the grafted RBE/NGF cells was observed.

3) Preparation of Tissues for Immunohistochemistry:

1 week, 3 weeks, 5 weeks, 8 weeks and 1 year after implantation, the animals are anaesthetized and perfused endocardially with 0.1 M PBS solution, pH 7.4 at 4° C., followed by a perfusion of 4% paraformaldehyde in the same buffer. The brains are removed and stored in the same buffer overnight at 4° C. The brains are then stored in PBS buffer comprising 30% sucrose for 2 days at 4° C. and frozen in isopentane at −60° C. Coronal and horizontal sections (thickness 30 μm) are cut using a cryostat and collected in wells filled with PBS at 4° C. The sections are divided into different groups in order to carry out an immunohistochemical analysis as well as toluidine blue staining.

For immunohistochemical analysis, the sections are initially treated with PBS containing 0.4% H₂O₂ for 30 minutes, and rinsed in the same buffer. They are then incubated in a 10% normal serum of the same animal as the one used to produce the secondary antibodies and 0.1% Triton X-100 in PBS for 1 hour, and thereafter with one of the following primary antibodies:

Polyclonal Primary Pntibody:

rabbit anti-glut-l (glucose transporter specific to the brain) antibody, (1:5000, Biogenesis); rabbit anti-GFAP antibody (1:6000, Dako); goat anti-ChAT antibody (1:100, Chemicon), rabbit anti-laminin antibody (1:5000, Sigma).

Monoclonal Primary Antibody:

mouse anti-p75 LNGFR (low affinity NGF receptor) antibody (1:150, clone 192, Boehringer); mouse anti-CD11b (rat macrophages) antibody (1:1000, clone MRC 0X-42, Serotec); mouse anti-rat T lymphocyte antibody (1:2000, clone MRC 0X-52, Serotec); mouse anti-rat major histocompatibilty complex I antibody (1:1000, clone MRC 0X-18, Serotec); mouse anti-rat MHC class II (Ia) antibody (1:1000, clone MRC -0X-6, Serotec); mouse anti-EBA (blood-brain barrier antigen specific to the rat) antibody (1:1000, clone SMI71, Affiniti).

All the antibodies are diluted in PBS buffer containing 5% of normal serum (donkey serum for the polyclonal antibodies and sheep serum for the monoclonal antibodies) and 0.1% Triton X-100, and incubated for 36 hours with stirring at 4° C. The sections are rinsed and incubated with biotinylated donkey anti-rabbit IgG (1:2000, Amersham) or anti-goat IgG (1:1000, Jackson Laboratories) antibodies or biotinylated sheep anti-mouse IgG antibodies (1:600, Amersham) in PBS buffer containing 5% of normal serum and 0.1% of Triton X-100, overnight with stirring at 4° C. They are rinsed, then incubated with a biotin-avidin-peroxidase complex (Vector Laboratories) for 30 minutes and rinsed again with Tris buffer (0.1 M TBS, pH 7.6). The sections are then incubated in a solution of diaminobenzidine tetrahydrochloride with nickel chloride and hydrogen peroxide (H₂O₂) in TBS buffer (0.05 M, pH 7.3). The enzymatic reaction is stopped by washing the sections in the buffer. The said sections are then counterstained and dehydrated, mounted on slides and observed under the microscope. A control is invariably carried out, by omitting the primary antibodies, and, under these conditions, the sections are always unlabelled.

4) Preparation of Tissues and Detection of the NGF Transgene by in situ Hybridization:

The cellular detection of βNGF transcripts in vivo is demonstrated by in situ hybridization of the NGF (MRNA) in the graft one week after implantation in the adult rat brain, as well as at 3 and 8 weeks, under the following conditions:

After deep anaesthesia with ketamine (150 mg.kg⁻¹, Imalgene), the rats are perfused with 2% paraformaldehyde in 0.1 M PBS buffer (pH 7.4, 4° C.). The brains are removed and placed in this buffer for 60 minutes at 4° C. After cryoprotection overnight in 15% sucrose solution in 0.1 M PBS at 4° C., rapid freezing of the samples is carried out by immersion in isopentane at −60° C. The frozen brains are cut horizontally (10-14 μm) using a Microm® cryostat, then mounted on gelatin-coated slides and dried at room temperature. The sections are prehybridized for one hour at 40° C. in 4×SSC, 1× Denhardt's buffer. Hybridization is carried out in a humid chamber at 37° C. for 16 hours, using as hybridization buffer a 4×SSC, 50% formamide, 10% dextran sulphate, 1× Denhardt's buffer, 500 μg/ml of fragmented and denatured salmon sperm DNA and 100 μg/ml of yeast tRNA mixture containing the abovementioned NGF probe at a final concentration of 2 μg/ml. The slides are washed sequentially in 2×SSC for one hour at 20° C., then in 1×SSC for one hour at 20° C., then in 1×SSC for half an hour at 37° C. and in 0.5×SSC for half an hour at 20° C. The digoxigenin-labelled, hybridized probe is detected using an immunoenzymatic detection kit (Boehringer-Mannheim) according to the manufacturer's instructions. Control procedures are carried out in parallel, either by digestion of the mRNAs with Rnase A (20μg/ml for 30 minutes at 37° C.), or by competition with an excess of unlabelled probe (excess of the order of 40) in the hybridization mixture. A dilution of the probe to 0.5 μg/ml gives a weak but specific signal. The absence of signal is observed when the NGF probe is not introduced during the hybridization.

In this case, a substantial presence of NGF transcripts is detected in the RBE/NGF grafts, reflecting a constitutive expression controlled by the LTR of this transgene.

FIG. 10 illustrates the in vivo study of the expression of the NGF transgene in RBE/NGF cells, three weeks after transplantation into the nucleus basalis (basal nucleus), and FIG. 11 illustrates the control brain structures used as internal control of the in situ hybridization of the NGF messenger, in vivo.

FIGS. 10A and 10B show a graft (G) of RBE/NGF cells strongly expressing the NGF transgene detected by in situ hybridization. This expression for the transgene still remains as strong 3 weeks after the intracerebral grafting. FIG. 10C visualizes a control graft (G) of uninfected RBE4 cells, grafted in the contralateral hemisphere, which does not display any positive NGF signal (×130 in 10A); (×270 in 10B and 10C, with transmitted interference contrast). FIG. 11A illustrates the neuronal detection of NGF in the frontoparietal cortex, and FIG. 11B illustrates the detection of NGF in the hippocampus (×260 in 11A); (×65 in 11B). These figures are in agreement with the established description of the endogenous synthesis of NGF by the neurons of the cerebral cortex and of the hippocampus in the adult rat.

5) Biological Effect of the NGF Produced by the Graft on the Cholinergic Neurons of the Basal Nucleus (Axonal Budding Effect):

To explore the biological effect of the NGF transgene product secreted in vivo, the following functional test is carried out: the modified cell line is grafted as specified above in the basal nucleus, in which the cholinergic neurons display a very sensitive response to NGF. These cholinergic neurons, apart from their expression of the enzyme ChAT, may also be characterized by a substantial immunoreactivity for the p75LNGFR receptor. The latter enables the cholinergic fibres and also their cell bodies to be visualized, especially during studies of axonal regeneration. The reactional budding detected by p75LNGFR immunoreactivity is observed up to at least three weeks in the RBE/NGF grafts.

In the first grafted group, the biological effect of the NGF produced by the grafts on the cholinergic neurons of the basal nucleus (axonal budding) is localized in this region and does not extend beyond the limits of the latter.

FIG. 12 illustrates the biological effect of the NGF secreted by RBE/NGF cells, three weeks after grafting, in the nucleus basalis (NB) (action on the promotion and maintenance of the reactive axonal regrowth of cholinergic neurons damaged after transplantation). In 12A, a general view of the shape of an RBE/NGF graft placed in the NB is visualized using the Hoechst prelabelling. In 12B, 12D and 12F, the effect of the NGF produced by the endothelial cells on axonal regrowth is visualized by the strong immunoreactivity of these axonal processes for the NGF p75 receptor. This axonal regrowth takes place over the entire length of the graft (G) and displays a strong reactivity around certain blood vessels (arrows).

In 12C and 12E, 3 weeks after grafting, control RBE4 grafts not infected with the NGF retroviral construction, placed in the NB of the contralateral hemisphere, are incapable of promoting and maintaining a reactive axonal regrowth of the cholinergic neurons of the NB (×65 in A, B, C, D, E and F, horizontal plane).

However, in the second group, the budding due to the NGF secreted by the grafted cells is more extensive in the ventrodorsal axis, along the graft, linking the basal nucleus to the dorsal striatum, thereby showing the trophic and tropic effects of NGF. FIG. 13 illustrates the biological effect of the NGF secreted by RBE/NGF cells, three weeks after grafting, away from the nucleus basalis, and illustrates the directional growth of the extensions in growth of the cholinergic neurons of the NB, along the graft up to the level of the dorsal striatum. FIGS. 13A and 13B illustrate a horizontal section passing through the dorsal portion of the graft in the striatum. In 13A, the RBE/NGF cells are visualized with the Hoechst nuclear stain. In 13B, the same section has been examined in transmitted light, showing a reactive axonal regrowth visualized with the anti-p75 NGF receptor antibody (×100 in 13A and 13B).

A quantification of the biological effect induced by the expression of the NGF transgene was undertaken according to the method described by Gundersen H. J. G. et al. (APMIS, 1988, 96, 379-394), by calculating the area occupied by the p75LNGFR immunolabelling at the sites of implantation of the RBE/NGF and RBE4 cells. The ratio of this area to that occupied by the graft was calculated at 3 and 8 weeks after implantation and is presented in FIG. 14, where the area occupied by -the p75LNGFR-positive structures (expressed as a percentage relative to the area of the graft) is plotted as ordinates.

Among the different clones tested in vivo, only the two move productive of NGF in vitro (clones RBE/NGF-2 and -4, mentioned above) gave rise to a biological effect in vivo, as summarized in Table III below:

TABLE III
Times after grafting

| | | | | |
|---|---|---|---|---|
| | 1 | 3 | 8 | 12 |
| clones | week | weeks | weeks | months |

| | | | | | |
|---|---|---|---|---|---|
| NGF4 | bio- | 0/5 | 5/5 | 1/5 | 0/2 |
| | logical | | | | |
| | effect | | | | |
| | NGF mRNA | 2/2 | 2/2 | 0/2 | |
| | Hoechst+/n | 5/5 | 5/5 | 5/5 | 2/2 |
| NGF4 | bio- | | 0/4 | | |
| (Thal- | logical | | | | |
| amus) | effect | | | | |
| NGF2 | bio- | 0/5 | 4/5 | 0/5 | 0/5 |
| | logical | | | | |
| | effect | | | | |
| | NGF mRNA | 2/2 | 2/2 | 0/2 | |
| | Hoechst+/n | 5/5 | 5/5 | 5/5 | 3/3 |

biological effect: number of brains where axonal growth was observed around the RBE/NGF graft relative to the total number of brains grafted.
NGF mRNA: number of brains where the NGF transcripts could be detected by in situ hybridization relative to the total number of brains treated.
Hoechst+/n: number of animals where Hoechst-prelabelled endothelial cells were detected relative to the total number of animals treated.

6) Immunological Tolerance:

In order to be able to study the host's immunological reaction with respect to the grafted endothelial cell lines, immunohistochemical labelling was carried out using markers of macrophages, of the major histocompatibility complex and of lymphocytes.

At one week, an infiltration of macrophages is observed at the transplantation site, with a decrease in their presence over time.

This infiltration is linked to the surgical trauma due to the transplantation.

However, no infiltration by lymphocytes is observed, even one month after transplantation.

These data suggest that such grafts are well tolerated by the host, which does not develop an acute rejection reaction with respect to the different endothelial cell lines grafted.

The above data show that both RBE4 cells alone and RBEZ cells and RBE/NGF cells survive and integrate after grafting. RBEZ and RBE/NGF cells are capable of expressing and/or secreting the product of the said transgene which, in the case of NGF, has the capacity to induce a biological effect in the brain.

As is apparent from the foregoing, the invention is in no way limited to those of its embodiments and modes of implementation and application which have just been described more explicitly; it embraces, on the contrary, all the variants which may occur to the practitioner in the field, without departing from the scope or compass of the present invention.

1. Mammalian endothelial cell lines, characterized:
in that they consist of immortalized endothelial brain cells displaying at least one of the following features of differentiated endothelial brain cells, in a stable manner:
the expression of endothelial markers,
the secretion of vasoactive substances,
the expression of molecules of the major histocompatibility complex (MHC),
the expression of hormone receptors, and
the existence of tight junctions,
in that they comprise a fragment of nucleic acid comprising at Least one immortalizing fragment of a viral or cellular oncogene, where appropriate in combination with at least one selectable gene, and an expression vector comprising a sequence coding for a polypeptide, a protein or a viral vector, where appropriate in combination with at least one selectable gene and where appropriate at least one reporter gene, and
in that they are capable in vivo of integrating in the brain vessels of a host mammal and of producing the said peptide, the said protein or the said viral vector.

2. Endothelial cell lines according to claim 1, characterized in that the nucleic acid fragment comprising at least one immortalizing fragment of an oncogene contains the neomycin resistance gene and a fragment of the SV40 T oncogene.

3. Endothelial cell lines according to claim 1, characterized in that the nucleic acid fragment comprising at least one immortalizing fragment of an oncogene contains the E1A early region of the adenovirus 2 genome and the neomycin resistance gene.

4. Endothelial cell lines according to any one of claims 1 to 3, characterized in that the said expression vector is a retroviral vector, in particular an MFG vector.

5. Endothelial cell lines according to claim 4, characterized in that the retroviral vector is an MFG-NB vector.

6. Endothelial cell lines according to any one of claims 1 to 5, characterized in that the sequence coding for a polypeptide or a protein is selected from the sequences coding for: enzymes, enzyme inhibitors, cytokines, neurotrophins, neurotransmitters, growth factors, toxins, antimetabolites, neurohormones, gangliosides, antibiotics, thrombolytic factors, coagulation factors, vasodilator or vasoconstrictor factors, hypo- or hypercholesterolaemic factors and hyper- or hypoglycaemic factors.

7. Endothelial cell lines according to any one of claims 1 to 6, characterized in that they consist of endothelial cells of brain capillaries.

8. Endothelial cell lines according to any one of claims 1 to 7, characterized in that they advantageously comprise, as immortalizing fragment, the E1A early region of the adenovirus 2 genome and the neomycin resistance gene, and, as expression vector, an MFG-NB retroviral vector containing the nls-lacZ gene coding for β-galactosidase.

9. Endothelial cell line according to claim 8, characterized in that it has been deposited under the No. I-1481 dated Oct. 10, 1994 with the Collection Nationale de Cultures de Micro-organismes [National Collection of Microorganism Cultures] held by the Pasteur Institute.

10. Endothelial cell lines according to any one of claims 1 to 7, characterized in that they advantageously comprise, as immortalizing fragment, the E1A early region of the adenovirus 2 genome and the neomycin resistance gene, and, as expression vector, a retroviral vector pMMuLVisisNGF coding for murine βNGF.

11. Endothelial cell line according to claim 10, characterized in that it has been deposited under the No. I-1482 dated Oct. 10, 1994 with the Collection Nationale de Cultures de Micro-organismes held by the Pasteur Institute.

12. Compositions, characterized in that they comprise at least one endothelial brain cell line according to any one of claims 1 to 11, in combination with at least one pharmaceutically acceptable vehicle.

13. Compositions according to claim 12, characterized in that they preferably contain between 10⁴ and 10⁵ endothelial cells/μl.

14. Method for obtaining a modified cell line according to any one of claims 1 to 11, which method is characterized in that:
(1) a first transfection is carried out by:
culturing endothelial brain cells, preferably those of brain microvessels, in a suitable culture medium supplemented with serum and with growth factors,
transfection of the said cells between the 2nd and the 6th passage with a nucleic acid fragment comprising at least one immortalizing fragment of a viral or cellular oncogene and, where appropriate, at least one selectable gene, in particular a gene coding for resistance to an antibiotic,
selection of the transfected cells on a selection medium suited to the said selectable gene, if necessary,
(2) a transfection of the cells obtained in (1) is then carried out with an expression vector containing the polypeptide sequence or protein sequence to be produced or a viral vector to be expressed.

15. Method according to claim 14, characterized in that the transfection of step (2) is carried out with a retroviral vector, preferably an MFG vector in which the sequence coding for the protein to be expressed has been incorporated beforehand.

16. Use of the mammalian endothelial cell lines consisting of immortalized endothelial brain cells, comprising a nucleic acid fragment including at least one immortalizing fragment of a viral or cellular oncogene, where appropriate in combination with at least one selectable gene, and where appropriate an expression vector comprising a sequence coding for a polypeptide, a protein or a viral vector, where appropriate in combination with at least one selectable gene and where appropriate at least one reporter gene, for obtaining a medicinal product for the treatment of primary and secondary neurological or psychiatric disorders or diseases, including brain tumours.

17. Use according to claim 16, characterized in that the said endothelial cell line is the line of immortalized endothelial brain cells which are deposited under the No. I-1142 with the Collection Nationale de Cultures de Micro-organismes.

18. Use according to claim 16, characterized in that the said endothelial cell line is an endothelial brain cell line according to any one of claims 1 to 11.

19. Application of the mammalian endothelial cell lines consisting of immortalized endothelial brain cells, comprising a nucleic acid fragment including at least one immortalizing fragment of a viral or cellular oncogene, where appropriate in combination with at least one selectable gene, and where appropriate an expression vector comprising a sequence coding for a polypeptide, a protein or a viral vector, where appropriate in combination with at least one selectable gene and where appropriate at least one reporter gene, for obtaining a medicinal product for stimulating the growth and reproduction of livestock.

20. Model for studying the integration in the brain of cells that deliver active substances to the brain, characterized in that it comprises a cell line according to claim 8 or claim 9.
